(19) United States
(12) Patent Application Publication
(10) Pub. No.: US 2001/0055589 A1
(43) Pub. Date: Dec. 27, 2001

(54) Cancer immunotherapy improved by prior radiotherapy

(75) Inventors: Henry M. Smilowitz, West Hartford, CT (US); Daniel N. Slatkin, Southold, NY (US)

Correspondence Address: ALIX YALE & RISTAS LLP, 750 MAIN STREET, SUITE 600, HARTFORD, CT, 06103

(73) Assignee: University of Connecticut

(21) Appl. No.: 09/931,440

(22) Filed: Aug. 16, 2001

Related U.S. Application Data
(63) Continuation-in-part of application No. 09/334,312, filed on Jun. 16, 1999, now Pat. No. 6,299,873.
(60) Provisional application No. 60/089,597, filed on Jun. 17, 1998.

(51) Int. Cl. A61K 48/00

(52) U.S. Cl. 424/93.7; 424/93.21

(57) ABSTRACT

A method of optimizing malignancy therapy includes surgical removal or reduction of the body burden of malignant cells and retention of some of those cells for subsequent use in immunotherapy. Radiotherapy may be employed at a radiation dosage below the threshold for normal tissue necrosis but on a correspondingly faster schedule than is the present United States standard to provide preservation of normal tissue structure. Normal tissue tolerable limits for one fraction are in the range of 10-13 Gy or Gy-Eq. The removed tumor cells are altered genetically to enhance their immunogenicity and also altered ex vivo by radiation or by any other means to render them incapable of sustained clonogenic propagation and then are re-injected using multiple sequenced injections.

### RELATED APPLICATION

This application is a continuation-in-part application of U.S. patent application Ser. No. 09/334,312, filed Jun. 16, 1999 which claimed the benefit under 35 U.S.C. § 119(e) of U.S. Provisional Patent Application Ser. No. 60/089,597 filed Jun. 17, 1998.

### BACKGROUND OF THE INVENTION

Typically, a patient with a malignant tumor undergoes preliminary therapy to greatly reduce the body burden of the malignant cells. This is conventionally accomplished by surgery. Depending on the clinical circumstances and on the type of malignancy, radiation therapy (“radiotherapy”) may be implemented after surgery or, less commonly, before surgery or even without surgery. Radiotherapy is usually administered to proven or putative tumor-bearing tissues. When macroscopically total or subtotal surgical excision is implemented, it is generally the practice to allow a 3 to 4 week postoperative period to elapse before the beginning of a course of fractionated photon-based radiation therapy, typically about 2 Gy per day, five days per week for about 6 weeks. However, radiotherapy can be useful if delivered at any time during the progression of the disease or even prophylactically, while clinical evidence of tumor progression is lacking.

Radiotherapy is usually accomplished using high-energy ionizing photons, typically gamma radiation, with energies measured in the millions of electron volts. It has been noted that there is an inadequacy of long-lasting efficacy of post-operative radiotherapy for human primary malignant brain tumors (often called ‘brain cancers’ or ‘malignant gliomas’), most of which are cerebral glioblastoma multiforme (GBM) in adults. Conventional photon therapy generally mandates delivery of as much radiation to the tumor as can be delivered, subject to the critical limitation that vital organs and tissues in and around the tumor that are in the path of one or several of the convergent photon beams receive doses that are below their thresholds for causing clinical dysfunction or for causing acute or delayed radiation-induced necrosis. In mainstream clinical radiation oncology, dose planning for conventional radiation therapy does not take into account thresholds for damage to the function or integrity of cells and tissues of the immune system that may be in the path of, or that indirectly may be adversely affected by, nominally therapeutic radiation.

Radiation therapy alone is often effective in slowing the growth of malignant tumors, but is usually incapable of preventing for much longer than one year devastating recurrence of deeply infiltrating growth of some types of malignancies, such as the most common primary malignant brain tumor, glioblastoma multiforme. Therapies for GBM, for example, are generally palliative until the final one-third of the total duration of postoperative survival. During the prior two-thirds, the patient is often in reasonably good functional health, albeit emotionally upset or even devastated by the knowledge of harboring a dreaded, latent malignancy.

If one attempted to use standard radiation therapy alone or after the surgical excision or partial excision of a malignant tumor in order to kill individual clonogenic tumor cells several centimeters beyond the macroscopic periphery of the tumor as imaged and/or visualized prior to surgery or radiotherapy, whether at its first occurrence or at its recurrence, such a large dose of radiation would be needed that normal brain structure and/or function would be compromised to some unacceptable degree. For example, long term (i.e., two years or more) survivors of glioblastoma multiforme not infrequently are left with significant neurological deficiencies attributed in part to the aggressive doses of radiation required to obtain such long survival. Nevertheless, it is known in the arts of cancer therapeutics that even well tolerated doses of standard radiation therapy do help to reduce by several orders of magnitude the number of brain tumor cells remaining after primary surgical extirpation (i.e., after surgical debulking) of the malignancy.

The present art of clinical radiation oncology uses standards for technique and doses that are unrelated to consideration of the function of the patient's immune system except in two circumstances: 1) where the therapy employs whole-body radiation; and 2) where partial or whole-body radiation follows or precedes chemotherapy. There is no method of immunotherapy (IMT) used and/or taught at present to treat malignant brain tumors or other malignant tumors that uses and/or teaches the necessity of implementing radiotherapy prior to, during, and/or after specific modulation and/or stimulation of the patient's immune system, and that the radiotherapy be of a certain advantageous quality for immunotherapy or immunoprophylaxis (IMPR) and that it be timed and given in dose levels to conform to the requirements of that specific immunotherapeutic modulation and/or stimulation.

Also, it is known that certain adjunct therapies, implemented after primary therapies, are useful in delaying and/or mitigating the regrowth of the cancer. Adjunct therapies known to be effective are chemotherapy, including antimitotic therapy and anti-angiogenesis therapy, additional radiation and further surgical removal or partial removal of the cancer.

It is readily discernible from the biomedical literature that experimental adjunct cancer therapies for humans based on gene therapy or immunotherapy are often, if not almost always, characterized by obvious insufficiency of analogy and/or of some major aspects of realism in the animal models of disease as compared to the clinical disease which the animal experimentation is intended to address. The most frequently encountered unrealistic features are: 1) inappropriate scale in dimensions of the tumor or numbers of malignant cells in the animal at the time of treatment and 2) the stage of advance of growth of the malignancy at the time of initiation of the experimental therapy.

Of the cytokines and other biochemical factors taught publicly as having shown effectiveness experimentally by using their genes to transfect cells ex vivo for gene-mediated immunotherapy (GMIMT) or gene-mediated immunoprophylaxis (GMIMPR), which, according to page 73 in A Primer of Brain Tumors: A Patient's Reference Manual, 25^{th} Anniversary Edition, 1998, American Brain Tumor Association, Des Plaines, Illinois, would be classified as either one or the other of two different kinds of “immune enhancer” gene therapy, none has ever been so identified in a clinically relevant animal model as explained above. Moreover, it has been taught that some such cytokines and factors are less effective than others or even that some are without any effectiveness at all for GMIMT or GMIMPR on the basis of tests using animals bearing tumors that are clinically unrealistic as explained above. A variety of cancer therapies dependent on the in vivo genetic modification of cancer cells have been proposed. Some of them are being tested in clinical trials. To date, none has shown to be sufficiently efficacious to warrant widespread adoption for common malignancies of the brain.

Although exposure of the brain cancer cells cultured in vitro to leukocytes obtained from the blood of a patient with a brain cancer can kill some of those cells, in vivo genetic transfection of sufficient numbers of brain cancer cells to cause the irreversible death of an entire human brain cancer, thereby stopping its otherwise deadly progressive growth, has not been previously achieved. There has been no gene delivery system that teaches a clinically practical technique to deliver a gene or several genes in vivo to a sufficient number of the potentially clonogenic brain cancer cells in a patient harboring a malignant primary brain cancer so that the delivery will enable full control of that brain cancer, whether that gene acts through inhibition of the growth potential of the brain cancer cells, through enhancement of the immunogenicity of those brain cancer cells, or through any other genetic modification of any cells in such a patient.

Some animal experiments that purport to demonstrate the efficacy of an immunotherapy technique for brain cancer fail to specify the state of advancement of the brain cancer in the test animals, which vitiate their claimed significance. Moreover, some inventors cite animal experiments that purport to demonstrate the inadequacy of an immunotherapy technique for brain cancer to highlight the efficacy of their own experimentally documented immunotherapy technique for brain cancer. However, such experimental studies are futile, however correct may be their experimental scientific formalism, if they do not take into account the absence of lymphatics in the brain and thus the relative inaccessibility of autologous brain tumor antigens to lymphoid tissues, the relative inaccessibility of lymphoid cells to some brain cancer tissues, and the requirement of ensuring that only a clinically relevant burden of experimental brain cancer cells be allowed to remain in amply vascularized brain cancer tissue in vivo at the time of initiation of the inoculations of antigen that characterize the designation “immunotherapy”.

For example, the experimental tumor can be far too small relative to the experimental animal organ under study to reasonably predict clinical relevance, since correlation in scale with the size of human tumors (at the time of clinical treatment) relative to the size of the human organ and/or organism is lacking. Additionally, in some instances, experimental immunotherapy or gene therapy is implemented at an inappropriate time, e.g., before the animal has time to manifest clinical symptoms or after tumor cells, modified in vitro, are transplanted to the test animals. The elimination of such tumors may be of scientific importance without necessarily having practical clinical relevance. A clinically relevant brain tumor may be deemed to be imminently lethal if it is so advanced that the residual life span of the untreated concomitant controls will be no more than one third of the total time between tumor inoculation and death from local tumor overgrowth in the brain. For example, the untreated 9L gliosarcoma causes death about three weeks after initiation. Clinically relevant experimental therapy may not begin until fourteen days after tumor inoculation. Anti-angiogenesis therapies are at too early a stage of clinical investigation to be evaluated in the context of this invention. However, any of such innovative therapies that proves clinically successful is likely to augment rather than degrade the effectiveness of the improvement in radiation therapy taught in this invention.

### SUMMARY OF THE INVENTION

The present invention combines the advantageous characteristics of these adjunct therapies while reducing or obviating such deficiencies as are indicated above. It involves a method of combining two disparate techniques, radiation therapy with immunotherapy or gene-modified immunotherapy (also called immunoprophylaxis when the treated malignancy is microscopic or clinically occult), whether with or without additional adjunct therapies, to effect a clinically useful synergy that would not exist if only one of these two modalities were implemented. Critical to this synergy is the expeditious implementation of extensive in vitro culture and, if appropriate, genetic transformation of the tumor cells as soon as possible after the first surgical debulking and, if feasible, before the start of daily radiation therapy treatments.

The present invention teaches that, although some tumor-sensitized leukocytes may be damaged or destroyed while circulating in the blood through the radiation field, some such cells will by chance not circulate there. Moreover, this invention teaches explicitly that the processes of initial sensitization of immune-reactive anti-tumor leukocytes and subsequent expansion of the numbers of such leukocytes circulating in the lymph and in the blood can best be promoted on a schedule that is primarily based on initiation as soon as possible after initial cancer surgery, limited mainly by consideration for the comfort of the patient receiving multiple subcutaneous sensitizing and boosting inoculations of ex vivo-transfected cancer cells, whether or not the surgical therapy is followed by radiation therapy. As a corollary, this invention teaches that the timely initiation of autologous tumor vaccination after initial tumor debulking should be considered particularly advantageous for brain tumors, as some subcutaneously injected brain tumor antigens are likely to be similar or identical to peptides derived from normal differentiation proteins, non-mutated cellular proteins, and/or mutated cellular proteins of the normal brain, thus not previously presented to the immune system as tolerogens.

This invention also teaches that the advantages resulting from the combination therapy of the present invention can, in certain patients, be augmented by significant (unconventional) reduction in the length of time required for the primary radiation therapy. Peripheral tumor nest cells are growing while conventional primary radiotherapy is implemented in small fractions daily over a period of several weeks (often six weeks). In one of its preferred embodiments, this invention prescribes use of a standard radiation therapy method that can be implemented by a technique of hyperfractionation, i.e., the administration of a dose of radiation (usually not more than 2 Gy per dose) several times per day, seven days per week, rather than once per day, five days per week for six consecutive weeks, as is now clinically standard in the United States.

Indeed, using rhesus monkeys as an experimental model for threshold doses of damage in the brain by hyperfractionated radiation, it has been shown by experimenters at the M.D. Anderson Cancer Hospital in Houston, Tex., that allowing about 6-8 hours between fractions is as effective in sparing that primate brain from damage as is allowing 24 hours between fractions. Thus, this invention also teaches in one of its embodiments that the presently recommended 30 fractions of post-neurosurgical radiotherapy could be as safely and effectively delivered in about 10 to 12 days as it is currently delivered in about six weeks, provided that the patient remained in or near the radiation therapy department during that 10 to 12 day period to receive one such fraction of about 1.7 to 2.0 Gy each every 8 hours.

The purpose of such hyperfractionation is to reduce the time interval between IMPR/GMIMPR-directed or IMT/GMIMT-directed expansion by the patent's immune system of tumor-killing circulating leukocytes (presently believed to be mixed populations of T-lymphocytes, natural killer lymphocytes, eosinophils, macrophages, and neutrophils) and the end of the course of temporally fractionated radiotherapy. Thus, using hyperfractionation, many tumor-killing leukocytes will escape being damaged by radiation while entering the tumor-bearing zone of the body. Also, any shortening of the total time (and thereby any increase in the intensity) of the tissue reaction to the radiation should prolong the transient breach of the blood-tumor barrier to passage of leukocytes from the circulating blood to the interstitial tumor tissue, and thereby augment the advantage to the patient of implementing radiotherapy in conjunction with IMT/GMIMT or IMPR/GMIMPR. The massive tumor necrosis that typically ensues after such rapid irradiation is believed to cause leukocyte diapedesis from local blood vessels due to a steep gradient of leukotaxic substances diffusing from the necrobiotic tumor mass. The large influx of leukocytes into the tumor area within a short time period will enhance tumor cell surveillance and therefore enhances the prospects of immunotherapy as compared with the slow influx of leukocytes associated with prolonged, less intensive radiotherapy.

The combination therapy of the present invention teaches restriction of the radiation dose to within a clinically tolerable range and advises but does not require precise spatial control of the radiation to limit the adverse effects on normal tissues thereof, whereby individual cancer cells beyond the periphery of the macroscopic tumor can be then be more effectively controlled by the infiltrating, tumor-killing leukocytes. The IMPR/GMIMPR or IMT/GMIMT can then target and be most effective against the residual microscopic nests of malignant tumor cells, the so-called “guerilla” cells, which should result in advantageous extension of symptom-free life.

Other objects and advantages will be in part obvious and in part pointed out more in detail hereinafter.

The present invention employs multi-modality therapy that combines a clinically tolerable dosage in radiation treatment to normal brain tissues, preferably over a short duration in conjunction with optimized immunotherapy. If appropriate and useful, the cells obtained from tumor surgery may or may not be irreversibly transfected so that tumor clones cultured in vitro are heightened in antigenicity and caused to acquire some properties different from those of their tumor cells of origin. As is well-known (for example, as stated in the review by Glenn Dranoff in Genetically Engineered Cancer Vaccines, Chapter 42, page 576 and in the references thereto cited by Dranoff on pages 578 and 579), in vitro irradiation is most frequently, but not always the technique used in the present art of clinical research on human cancer vaccines to generate live, growth-arrested tumor cells for cancer vaccines.

Experimentation has shown that, for two different rat brain tumor cell lines growing in vitro, 50 to 70 Gy of ionizing photons (e.g., gamma rays) given in one dose in less than one hour will suffice to destroy all clonogenic capability. Thus, cells cultured under sterile conditions may or may not be lethally irradiated before injection as a vaccine into the patient. The cells are re-injected to elicit either a heightened natural or a new iatrogenic immune response to the primary cancer cells as well as to themselves. In its preferred embodiment, this invention is implemented in conjunction with any ancillary cancer therapy that slows tumor growth e.g., antiangiogenic therapy, dendritic cell/chimeric cell-mediated immunotherapy, antisense or virosome/virus mediated gene therapy, selective enzyme-inhibition therapy such as anti-PDE subtype therapy, antagonism of endogenous growth factor therapy or bacterial therapy.

### BRIEF DESCRIPTION OF THE DRAWINGS

The present invention may be better understood and its numerous objects and advantages will become apparent to those skilled in the art by reference to the accompanying drawings in which:

FIG. 1 is a Kaplan-Meier plot comparing the fraction of surviving rats as a function of the number days after therapy of a first control group of rats, a second group of rats treated with radiation only, and a third group of rats treated with a combination of immunoprophylaxis and LINAC-based photon therapy and

FIG. 2 is a Kaplan-Meier plot illustrating the fraction of surviving rats as a function of the number days after therapy of a first control group of rats, a second group of rats treated with radiation only, and a third group of rats treated with a combination of gene mediated immunoprophylaxis and LINAC-based photon therapy.

### DETAILED DESCRIPTION OF THE PREFERRED EMBODIMENT

As mentioned above, the present invention involves a method of optimizing malignancy therapy using post-operative radiation and immunotherapy techniques developed using imminently lethal rat brain tumors, unlike the relatively minuscule murine tumors that are showcased in virtually all of the preclinical studies published heretofore. It will be appreciated that certain malignant tumors may be located where surgical treatment alone or in combination with adjunct radiation or immunotherapy will provide a satisfactory result. However, it is also known that many tumors are located in areas where adjacent sensitive tissue will be placed in jeopardy by such therapies. Therefore, for purposes of illustration and ease of understanding, the invention will be described in connection with primary malignant brain tumors that are situated in or near neurologically eloquent normal brain structures. It will be understood that tumors located in extracranial areas such as those in or near the spinal cord and especially but not exclusively, those in and near vital tissue structures of the neck, where high levels of radiation might damage adjacent organs and tissues, will also benefit from this technique.

It also will be appreciated that while the present invention envisions the surgical removal or reduction of the body burden of the malignant cells, in certain cases surgery may not be feasible or are predicted to be irreversibly and intolerably harmful to body functions. Then, a small portion or a minuscule biopsy of the tumor may be the only tissue obtainable to implement the gene-mediated immunotherapy portion of the combination treatment described herein, which would however not in any material sense vitiate the clinical effectiveness of this invention.

Following preliminary surgical removal of a cancer to greatly reduce the body burden of cancer cells (or, if necessary, following only surgical or needle biopsy of the cancer), radiation therapy is implemented in the brain. The standard for conventional radiation doses, while below the thresholds for acute and delayed radiation-induced brain necrosis, typically does not take into account damage to the function and integrity of cells and tissues of the immune system. In accordance with the present invention, the radiation dosage must be below the threshold for normal tissue necrosis and must provide not only for the preservation of normal tissue structure but also for maintaining and enhancing tumor immunosurveillance. Therapies of this type could include proton therapy, hyperfractionation or standard fractionation of either conformal (with or without amplitude modulation) or conventional electron linear accelerator-mediated (or so-called LINAC-mediated) megavoltage photon radiotherapy, synchotron-generated x-ray microbeam therapy, ultra-high energy heavy-ion therapy, photodynamic therapy, and radioisotope-mediated brachytherapy, and boron neutron20 capture therapy.

Experience from fast-neutron therapy-mediated ablation of those kinds of human brain tumors called glioblastoma multiforme (GBM), a subtype of malignant astrocytoma (in early clinical trials, that inadvertently delivered supra-threshold doses of radiation to contiguous normal brain tissues), revealed that ablation of GBM would require at least 30 Gray (Gy) if delivered in a single fraction (gray is the MKS unit of physical absorbed dose representing the delivery of ionizing radiation energy to a final concentration of one joule per kilogram of tissue) of photon (x-ray or gamma-ray) radiation. But a single dose of photons of that magnitude would seriously injure the normal brain, the normal brain tissue tolerance limit being only about 12-13 Gy in a single fraction.

In general, it is believed that adult human central nervous system tissue cannot tolerate much more than 10-13 Gy of radiation in a single session to an appreciable fraction of the volume of the central nervous system without serious short-term or long-term adverse effects. Thus, the method of immunotherapy described herein can be used in conjunction with any kind of single-fraction radiotherapy (e.g., gamma-based radiosurgery; boron neutron-capture therapy; brachytherapy using high dose-rate, removable radioisotope-loaded “seeds”) in which the dose to normal brain endothelium is raised to its tolerable maximum (probably in the range 10-13 Gy of photon radiation (low-LET radiation) or in the range of doses of more densely ionizing radiations (high-LET radiation) equivalent in their therapeutic effects to those of 10-13 Gy of photons, i.e., 10-13 Gy-equivalent [Gy-Eq]), given to an appreciable fraction of the ipsilateral brain hemisphere's volume) while increasing the minimum dose to the brain tumor region to at least 30 Gy-Eq. Surgery and radiotherapy can primarily ablate the major tumor mass and then IMPR/GMIMPR or IMT/GMIMT can primarily target the sparsely cellular microscopic nests of tumor cells beyond the periphery of the main tumor. The numerical parameter for what would be now considered ideal radiation doses and radiation therapy schedules to implement this invention may, with clinical experience, be found somewhat different from those suggested herein. Nevertheless, the invention would not be vitiated by any such adjustments in dose.

In its preferred embodiment, the immunotherapy should be such that the subcutaneously or intradermally injected “immunogens” stimulate the clonal expansion of therapeutically effective subsets of leukocytes, especially cytotoxic T cells, directed toward ablation of the residual cells of the neoplasm long after surgery and/or radiotherapy.

Immunotherapy will benefit from a substantial flux of immune cells throughout the targeted tumor and should be most effective with the smallest possible residual tumor burden. Immunotherapy is implemented beginning immediately after or, if appropriate, before the end of radiation therapy. Typically, radiation therapy occurs within four weeks after surgery, and immunotherapy is implemented within ten weeks after surgery, but variations from this schedule, great or small, should not materially vitiate the efficacy of immunotherapy, e.g., gene-mediated immunotherapy, using cells cultured in vitro that are genetically identical to or genetically similar to cells of the cancer being treated. Most likely, but not necessarily, they may have to be irreversibly transfected, i.e., genetically transformed, so that the transfected cells and the progeny or clones of the transfected cells will have either membranes that are subtly different from those of their cells of origin in the patient's tumor or have biochemical properties that are subtly different from those of their cells of origin. For example, the clones may lack either one or several growth factor receptors, growth factors themselves, or a combination of growth factor receptors and growth factors.

The clones will have altered levels of molecules needed to enhance their immunogenicity and/or ability to stimulate (immunemediated) tumor cytotoxicity. For example, these modifications will be brought about by antisense gene modification, by fusion with dendritic cells, by transfection with CD80, CD86, or MHC, or by any technique found to be effective in stimulating tumor cell antigenicity. The clones are then cultivated in vitro en masse so as to provide a large number (at least several million per injection) of identical, genetically modified tumor cells. These living cells, grown in sterile culture media, are then either used as such or processed (by supralethal doses of radiation or by other means) to render them incapable of unlimited clonogenic propagation in vitro or in vivo.

Unmodified tumor cells themselves or the genetically modified tumor cells, membrane fragments of these cells, (suspended in a suitable medium, preferably with an immunogenic adjuvant, e.g., Freund's adjuvant), or specific antigenic determinants extracted or derived from those cultured tumor cells are used for injection subcutaneously, intradermally, or intramuscularly into the patient using standard sterile techniques, such injections being repeated as often and as many times as may be shown beneficial by the future clinical applications of this invention. The whole cells or cell fragments so injected then elicit a response in the patient characterized by increased immunological surveillance and recognition of the primary cancer cells and their progeny in the patient as non-self (i.e., as foreign cell types) so as to confer to the patient an increased ability to reject, i.e., kill, any residual and/or recurrent cancer cells weeks, months, or even years after the initial therapy of the malignancy.

The present invention circumvents many of the clinical inadequacies mentioned hereinbefore by using gene modification methods in vitro to produce gene-modified tumor cells that, in conjunction with normal-tissue-sparing radiotherapy such as proton therapy and the like, will stimulate, expand, and mobilize tumorcidal cells of the patient's own immune system in vivo. Additionally, this invention discloses the use of clinically relevantly-sized brain tumors experimentally, i.e., about 40 mg±20 mg tumors in the mature rat brain, occupying several percent of the test rat cranium for use in preclinical gene-mediated immunotherapy experiments in rats. The current state of the art of experimental immunotherapy of brain tumors generally uses rodent tumors (usually murine tumors) that are considerably smaller in proportion to the size of the animal brain in which they are being tested than is the case for almost any symptom-evincing human brain malignancy diagnosed in vivo.

A feature of this invention is that the immunological stimulation of the patient may be implemented in such a manner that the body burden of cancer cells that remains after radiotherapy is maximally subjected to immunological rejection when that burden is minimized by radiation therapy. For example, the brain tumor cells may first be lethally irradiated in vitro to thereafter produce a transiently growing, then spontaneously regressing subcutaneous neoplasm. Alternatively, live, clonogenic tumor cells cultured in vitro that lack the biological mechanism for their widespread systemic dissemination in vivo (i.e., for their metastasis) may be injected subcutaneously and the resulting tumors removed surgically if they do not regress spontaneously beforehand. If appropriate, the live or irradiated tumor cells may be injected in multiple microscopic jets of fluid using an apparatus similar to that known in veterinary medicine as the pig jet to maximize the efficiency with which tumor cells interact with the Langerhans cells of the lower epidermis and subcutaneous tissues which in turn will migrate as “veil” cells or dendritic cells in the lymph to the paracortical region of lymph nodes to interdigitate with and stimulate appropriate precursors of cytotoxic T cells.

The present invention provides a combination of single-fraction or several-fraction postoperative radiation therapy with re-injection of cells or cell components derived from in vitro culture of the patient's own brain tumor cells. The tumor cells may or may not be modified by genetic transformation during cell culture in vitro and re-injected into the same patient at or shortly after the initial neurosurgical removal of the bulk of the tumor (i.e., after the first debulking). Neither of these two therapies implemented without the other will be as effective as the combination of the two because 1) radiation doses are inevitably limited by concomitant radiation damage to normal tissues and 2) the immune response to residual and regrowing tumor cells, no matter how specific, will be limited by the small fraction of the total cytocidal and natural killer T-lymphocyte population that can be not only induced to tumorcidal specificity in the human body but also induced to accumulate in and around tissues near the margins of a neurosurgical scar. There are only about one billion new lymphocytes produced per day in human adults. Thus, it is unlikely that a residual human tumor burden of more than a few percent of one billion cells could be ablated in time to prevent regrowth of a malignant brain tumor. The recently publicized developments of immunotherapy for malignant gliomas focus largely on achievement of immunospecificity in small animal tumor models, with little or no consideration given to quantification of the speed and intensity of an immune response in the animals that would be relevant to clinical efficacy in man.

The great advantage of a radiosurgical (single-fraction) or hyperfractionation (several fractions per day) modality of radiotherapy is that the infiltration of radiation-sensitive lymphocytes into the surgically disturbed tissue is expected to be largely unimpeded or, at worst, delayed by several days on account of the radiation. The induction of such infiltration by subcutaneous injections of cells is best begun at the earliest possible time after surgical debulking, as the efficacy of those injections might be inhibited or somewhat vitiated if they were begun after or shortly before the termination of the protracted postoperative course of once-daily radiation treatments carried out in most postoperative brain tumor primary radiotherapy practiced to date. The particular kind of in vitro tumor cell transfection exemplified herein simply illustrates the kind of transfection that can be used to heighten the antigenicity of the patient's tumor cells. Likewise, the particular kind of radiotherapy employed not only is of a kind that minimally disturbs the influx of tumor-interactive leukocytes into the irradiated tissues, but also exemplifies the kinds of radiotherapy likely to induce, by themselves, such long-lasting suppression of tumor growth that there should be ample time for iatrogenic tumor-cell immune rejection reactions to take place at or near the site of previous tumor debulking, i.e., in tissues at greatest risk for tumor recurrence.

As a specific example of the invention, gene-mediated immunotherapy of glioblastoma multiform (GBM) is implemented immediately after proton therapy (PT) or, in some cases, perhaps before PT but after surgical biopsy or debulking of the tumor. In the former case, on the day (or on the last day) of PT, one or several subcutaneous injections of the gene-modified glioblastoma cells are given to the patient. Booster injections of the same or similar immunogenic mixtures would be given at regular or irregular intervals thereafter, typically at two-week or at one-month intervals. The patient's clinical status would be assessed by a physician's direct examination of the patient and by noninvasive imaging, typically by magnetic resonance imaging, at intervals of about one month. As a result, both the length and quality of life are improved. Heightened immunological surveillance, recognition, and rejection of residual viable clonogenic GBM cells will lengthen the interval between treatment and clinically detectable recurrence as well as the interval between treatment and death from intractable overgrowth of the GBM in the brain.

### EXAMPLES

The untreated 9L gliosarcoma (9LGS), a well established, demonstrably immunogenic, malignant rat brain tumor cell line, causes death within 21±3 days (mean ±SD) after the intracerebral implantation of about 10⁴ cultured cells in one microliter of culture medium in isogenic rats. Clinically relevant therapeutic intervention was not begun until 14 days after tumor inoculation, at which time the relative tumor volume approximates that of human GBM at diagnosis and the tumor is so advanced that the median residual life span of the concomitantly untreated control animals is no more than one third of the total time between tumor inoculation and death from tumor overgrowth. Experiments utilizing rats have shown that immunization with a series of subcutaneous injections of radiation-killed unmodified 9LGS cells (immunoprophylaxis or IMPR) initiated on day 14 after tumor inoculation, not followed by irradiation of the tumor, is therapeutically ineffective. However, the same regimen becomes highly effective if preceded by deliberately sub-optimal boron neutron-capture therapy (BNCT), a binary treatment modality that can selectively irradiate tumor tissue.

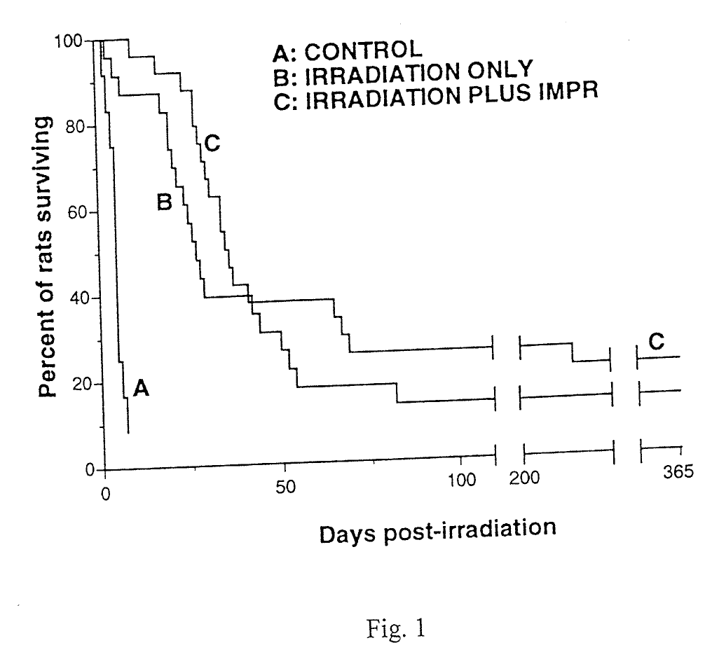

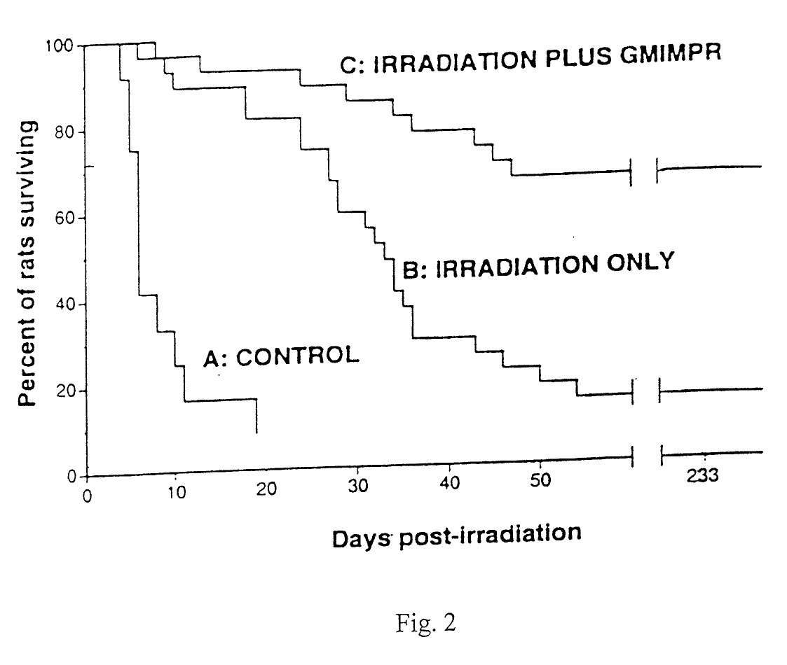

Most radiotherapy of human GBM is mediated by megavoltage photons. Accordingly, experiments were conducted to test the combination of IMPR with megavoltage photon irradiation of relatively large, clinically relevantly-sized, imminently lethal, intra-cerebral rat 9LGS experimental brain tumors. Such experiments revealed that, unlike the combination of BNCT and IMPR with unmodified radiation-disabled 9LGS cells, which is decidedly effective, the combination of single-fraction photon irradiation and IMPR is only marginally effective (FIG. 1). In sharp contrast, the combination of LINAC-mediated 6 MeV photon therapy and GMCSF gene-mediated IMPR (GMIMPR) is highly effective (FIG. 2).

The Combination of Immunoprophylaxis (IMPR) and LINAC-based 6 MeV Photon Therapy for Advanced Rat Brain Tumors.

In the experiments combining single-fraction photon irradiation and IMPR, Fischer 344 rats received an intra-cerebral (ic) injection of approximately 1 μl of cell culture medium containing 10⁴ 9LGS cells.

Fourteen days later (day 0), a first group of thirteen rats were left untreated as a control, a second group of twenty seven rats were treated with radiation only (given as a single fraction, 25 Gyₘₐₓ), and a third group of twenty seven rats began treatment with radiation plus multiple injections of 5×10⁶ in vitro-irradiated (50 Gy) 9LGS cells. The third group of rats was re-injected with similarly irradiated 9LGS cell on day 7, day 21, and every two weeks thereafter. A survival analysis using the log rank test shows that the number of long-term surviving rats that received LINAC irradiation plus immunoprophylaxis with unmodified 9LGS cells is not significantly greater than the number of long-term surviving rats that received LINAC irradiation only. Specifically, if the curve for the combination of LINAC irradiation and IMPR is superimposed on the curve for LINAC irradiation-only, the two curves merge, disclosing that there is no statistically significant difference between the two groups.

The Combination of GMIMPR and LINAC-based 6 MeV Photon Therapy for Advanced Rat Brain Tumors.

In the experiments combining LINAC-based 6 MeV photon therapy and GMCSF GMIMPR, Fischer 344 rats received an intracerebral (ic) injection of approximately 1 μl of cell culture medium containing 10⁴ 9LGS cells. Fourteen days later (day 0), a first group of thirteen rats were left untreated as a control, a second group of twenty seven rats were treated with radiation only (given as a single fraction, 25 Gyₘₐₓ), and a third group of twenty seven rats began treatment with radiation plus multiple injections of 5×10⁶ in vitro-irradiated (50 Gy) GMCSF-transfected 9LGS cells. The third group of rats was re-injected with irradiated GMCSF-transfected 9LGS cell on day 7, day 21, and every two weeks thereafter. A survival analysis using the log rank test shows that the number of long-term surviving rats that received LINAC irradiation plus gene-mediated immunoprophylaxis is significantly greater than the number of long-term surviving rats that received LINAC irradiation only (p<0.0007) and significantly greater than the number of long-term surviving rats that received the combination of LINAC irradiation plus immunoprophylaxis with unmodified, irradiated 9LGS cells (p<0.001).

The experimental results indicated that GMCSF stimulates immune system function by enhancing the presentation of antigens by the antigen presenting cell (APC) to the T lymphocyte. GMCSF had been identified in non-radiotherapeutically relevant screening tests by others to be the most potent inducer of antitumor immunity out of the large number of immunomodulators screened.

The GMCSF transfectant utilized in the subject experiments was a rat GMCSF expression vector, in which rat GMCSF PCR product was cloned into the eukaryotic expression vector pCR3 (Invitrogen). The GMCSF transfectant was provided by Dr. Martin Oaks, University of Wisconsin Medical School, Milwaukee, Wis. (Oaks, M. K., Penwell, T., Suh, C. -H. & Tector, A. J. (1 995) J. of Interferon and Cytokine, Res. 15, 1095-1102). Transfections were performed using the Eukaryotic Transfection Kit (Stratagene Corp., La Jolla, Calif.). GMCSF transfectants were screened by the ability of cell culture supernatants to support the growth of FDC-P1 cells (American Type Culture Collection), which require GMCSF for their growth and viability. Transfectants were assessed by collecting supernatants after 24 hours of post-confluent culture and comparing them with known quantities of recombinant rat GMCSF for their ability to support the growth of FDC-P1 cells (Oaks, M. K., Penwell, T., Suh, C. -H. & Tector, A. J. (1995) J. of Interferon and Cytokine, Res. 15, 1095-1102). Robust transfectants, producing 5-0200 ng GMCSF/10⁶ cells/24 hours, were subcloned. The subclone 9LGMCSF 27-1 (approximately 150 ng GMCSF/10⁶ cells/24 hours) was used for these experiments and retested after the experiments as described above and, independently, by an ELISA-based technique, for its ability to robustly produce GMCSF. Concomitantly, those two tests were used after the experiments to confirm that the parent 9LGS cell strain produced no detectable GMCSF.

The subclone, 9LGMCSF 27-1 was cultured in DMEM supplemented with 10% fetal bovine serum (Hyclone), Penicillin/Streptomycin (100 U/100 μg per ml), L-Glutamine (2 mM), fungizone (0.25 μg/ml) and geneticin (1 μg/ml) (GIBCO). The cells were seeded on T75 and T175 flasks (Sarstedt) (˜1.5×10⁶ and ˜3×10⁶, respectively) such that confluence was achieved in about one week. The lowest-passage cells were frozen in multiple aliquots and periodically thawed, such cells were not used for immunizations beyond passages. Rats were injected subcutaneously in the left thigh with 5×10⁶ cells immediately following radiosurgery, seven days later and every two weeks thereafter for a minimum of 4 months in some experiments and up to 1 year in other experiments. Prior to harvesting the cells for injection, the cell culture medium was harvested and assayed for FDC-P1 growth promoting factor as described above.

The experiments demonstrated that the combination of IMPR with BNCT and the combination of GMIMPR with LINAC-based photon therapy are both effective therapeutic combinations for the treatment of advanced brain tumors in a mammal. Although not explicitly proven, it is reasonable to infer that the combination of BNCT and GMIMPR would also be effective since BNCT and IMPR is effective and GMIMPR is a more powerful form of immunoprophylaxis than is IMPR.

While preferred embodiments have been shown and described, various modifications and substitutions may be made thereto without departing from the spirit and scope of the invention. Accordingly, it is to be understood that the present invention has been described by way of illustration and not limitation.

What is claimed is:

1. A method of treating a malignant solid glioma in a human or animal using a multi-modality therapy, the solid glioma defining a parental tumor strain, the therapy comprising the steps of:
(a) surgically excising cells of the solid glioma;
(b) altering said excised cells by genetic modification to enhance the immunogenicity of the altered cells;
(c) subjecting the solid glioma to a radiation therapy method while limiting the radiation dose received concomitantly by contiguous normal tissue to clinically tolerable levels; and
(d) subjecting said human or animal to an immunotherapy composed of introduction of said altered cells into said human or animal using multiple sequenced injections.

2. The treatment method of claim 1 wherein the mass of the solid glioma is at least substantially reduced prior to step (a) using surgical techniques.

3. The treatment method of claim 1 wherein the mass of the solid glioma is at least substantially reduced prior to step (a) using radiotherapeutic techniques.

4. The treatment method of claim 1 wherein the mass of the solid glioma is at least substantially reduced prior to step (a) using surgical and radiotherapeutic techniques.

5. The treatment method of claim 1 wherein the radiation dose received by normal tissue adjacent the solid glioma is in the range of 10-13 Gy from low-LET radiation or a total of 10-13 Gy-Eq from any combination of low-LET and high-LET radiations.

6. The treatment method of claim 1 wherein step (b) includes lethally irradiating the cells in vitro to render them incapable of sustained clonogenic growth in vivo.

7. The treatment method of claim 1 wherein step (c) includes subjecting the solid glioma to LINAC radiation.

8. The treatment method of claim 1 wherein the altered cells are cultivated to provide a large number of identical, genetically modified cells for use in step (c).

9. The treatment of claim 1 wherein the altered cells of step (b) are mixed in an immunogenic adjuvant suited for injection in step (c).

10. The treatment method of claim 1 wherein step (c) is implemented upon completion of step (a).

11. The treatment method of claim 1 wherein the radiation therapy is completed in less than a few hours after completion of step (a).

12. The treatment method of claim 1 wherein the radiation therapy is completed in less than a few days after completion of step (a).

13. The treatment method of claim 1 wherein the radiation therapy is completed within fewer than ten weeks after completion of step (a).

14. A method of treating a malignant solid glioma in a human or animal using a multi-modality therapy comprising the steps of:
(a) surgically excising cells of the solid glioma;
(b) genetically modifying said excised cells to enhance the immunogenicity of said genetically modified cells;
(c) altering the genetically modified cells to render the altered cells incapable of sustained clonogenic propagation;
(d) subjecting the solid glioma to LINAC radiation therapy while limiting the radiation dose received concomitantly by contiguous normal tissue to clinically tolerable levels; and
(e) subjecting said human or animal to an immunotherapy composed of introduction of said altered cells into said human or animal using multiple sequenced injections.

15. A method of treating a malignant solid glioma in a human or animal using a multi-modality therapy comprising the steps of:
(a) surgically excising cells of the solid glioma;
(b) genetically modifying said excised cells to enhance the immunogenicity of said genetically modified cells;
(c) altering the genetically modified cells to render the altered cells incapable of unlimited clonogenic propagation;
(d) subjecting the solid glioma to LINAC radiation therapy while limiting the radiation dose received concomitantly by contiguous normal tissue to clinically tolerable levels; and
(e) subjecting said human or animal to an immunotherapy composed of introduction of said altered cells into said human or animal using multiple sequenced injections.
